(19) United States
(12) Patent Application Publication
(10) Pub. No.: US 2024/0246084 A1
(43) Pub. Date: Jul. 25, 2024

(54) APPARATUS INCLUDING A LIQUID COOLANT PASSAGE FOR AMPLIFICATION OF A NUCLEIC ACID

(71) Applicant: Hewlett-Packard Development Company, L.P., Spring, TX (US)

(72) Inventors: Carson Denison, Corvallis, OR (US); Alexander Govyadinov, Corvallis, OR (US); Richard W. Seaver, Corvallis, OR (US); Erik D. Torniainen, Corvallis, OR (US); Pavel Kornilovich, Corvallis, OR (US); Anand Samuel Jebakumar, Corvallis, OR (US)

(73) Assignee: Hewlett-Packard Development Company, L.P., Spring, TX (US)

(21) Appl. No.: 18/559,761

(22) PCT Filed: May 10, 2021
(86) PCT No.: PCT/US2021/031650

§ 371 Date: Nov. 8, 2023

(51) Int. Cl. B01L 7/00

(52) U.S. Cl. CPC B01L 7/52; B01L 2300/1805; B01L 2300/1894

(57) ABSTRACT

Apparatuses may be used for nucleic acid amplification. An example apparatus may include an amplification chamber to contain a biochemical reaction associated with amplification of a biologic sample. The biologic sample may include a nucleic acid. The apparatus may also include a heater thermally coupled to the amplification chamber. The apparatus may also include a thermally conductive substrate in contact with the heater and including a liquid coolant passage to pass a liquid coolant through the thermally conductive substrate.

### BACKGROUND

Nucleic Acid amplification, of which Polymerase Chain Reaction (PCR) is one example, is used in molecular biology to make many copies of a nucleic acid segment. Using PCR, a single copy (or more) of a nucleic acid sequence is exponentially amplified to generate hundreds of millions or more copies of that particular nucleic acid segment. Many PCR methods rely on thermal cycling. Thermal cycling methods expose reactants to repeated cycles of heating and cooling to permit different temperature-dependent reactions to occur.

### BRIEF DESCRIPTION OF THE DRAWINGS

FIG. 1 illustrates an example apparatus including a thermally conductive substrate including a liquid coolant passage for amplification of the nucleic acid, in accordance with the present disclosure.

FIG. 2 illustrates an example apparatus including a thermally conductive substrate including a liquid coolant passage for amplification of the nucleic acid and further illustrating a fluid inlet and a fluid outlet for the liquid coolant passage, in accordance with the present disclosure.

FIG. 3 illustrates an example apparatus including a thermally conductive substrate including a liquid coolant passage for amplification of the nucleic acid in which a reagent input/output and a liquid coolant input/output are on a same side of the apparatus, in accordance with the present disclosure.

FIG. 4 illustrates an example apparatus including a thermally conductive substrate and a plurality of liquid coolant passages for amplification of a nucleic acid, in accordance with the present disclosure.

FIG. 5 illustrates an example apparatus including a thermally conductive substrate and a plurality of liquid coolant passages for amplification of a nucleic acid, in accordance with the present disclosure.

FIG. 6 illustrates an example apparatus including a thermally conductive substrate and a plurality of amplification chambers for amplification of a nucleic acid, in accordance with the present disclosure.

FIG. 7 illustrates an example apparatus including a thermally conductive substrate, a plurality of amplification chambers for amplification of a nucleic acid, and liquid coolant inlet troughs, in accordance with the present disclosure.

FIG. 8 illustrates an example apparatus including a plurality of amplification chambers for amplification of a nucleic acid and a venting port, in accordance with the present disclosure.

FIG. 9 illustrates an example apparatus including a jetting nozzle-venting port, in accordance with the present disclosure.

FIG. 10 illustrates an example method for amplification of a nucleic acid, in accordance with the present disclosure.

FIG. 11 illustrates a block diagram of an example apparatus used during a feasibility experiment.

FIG. 12 illustrates peak heat fluxes to the amplification fluid at 162, 165, and 188 mW for liquid coolant flows, in accordance with the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

The Polymerase Chain Reaction (PCR) is a method for multiplication and subsequent detection of DNA sequences. In order to perform one duplication of a nucleic acid sequence, the sample temperature is raised to approximately 95° Celsius (° C.), cooled to approximately 55° C., and held at approximately 75° C. PCR may also be performed by cycling between two temperatures; a high temperature ranging between approximately 90-95° C., and a low temperature ranging between approximately 65-70° C. To amplify a segment of deoxyribonucleic acid (DNA) to detectable levels, this thermal cycle may be performed 20-40 times. As such, PCR reaction times can take up to a few hours to complete depending on the quality and concentration of the target nucleic acid. This cycle time cannot meet the demand of many current genetic amplification applications.

Often, the rate limiting step for a PCR reaction is the speed of thermal cycling. In accordance with the present disclosure, a thermally conductive substrate including a liquid coolant passage for amplification of a nucleic acid is described, which allows for rapid cooling of biologic samples and thereby reduction of cycle times for nucleic acid amplification. Example devices described herein have the potential to perform a PCR cycle in under one second with the total reaction time of less than 30 seconds.

In accordance with the present disclosure, an example apparatus comprises an amplification chamber to contain a biochemical reaction associated with amplification of a biologic sample including a nucleic acid, a heater thermally coupled to the amplification chamber, and a thermally conductive substrate in contact with the heater and including a liquid coolant passage to pass a liquid coolant through the thermally conductive substrate.

In accordance with another example of the present disclosure, an apparatus comprises an amplification chamber to contain a biochemical reaction associated with amplification of a biologic sample including a nucleic acid, a series of heaters thermally coupled to the amplification chamber, and a thermally conductive substrate in contact with the series of heaters and including a liquid coolant passage to pass a liquid coolant through the thermally conductive substrate.

In accordance with another example of the present disclosure, a method comprises receiving at an amplification chamber defined by a thermally conductive substrate, a biologic sample including a nucleic acid, heating a heater thermally coupled to the amplification chamber to a temperature of a heating and cooling protocol, the heating and cooling protocol associated with amplification of the nucleic acid, receiving at a liquid coolant passage defined by the thermally conductive substrate and in contact with the amplification chamber, a liquid coolant, and cooling the biologic sample for an amount of time associated with the heating and cooling protocol.

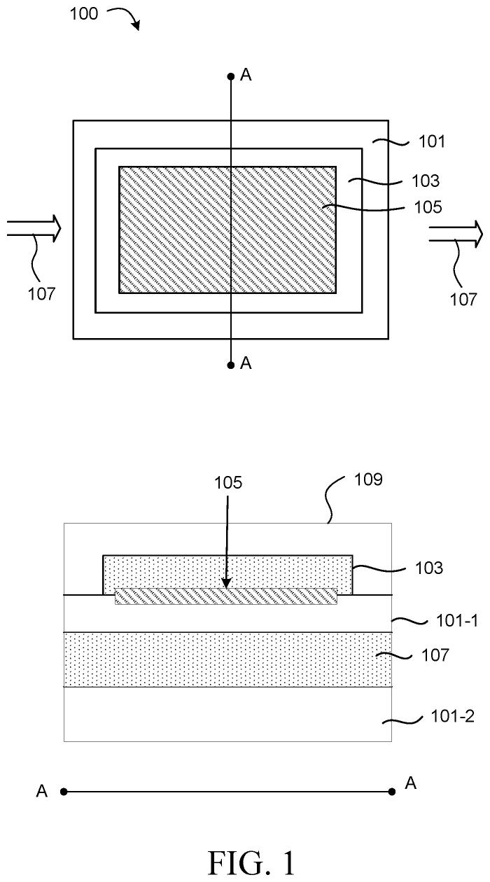

Turning to the figures, FIG. 1 illustrates an example apparatus 100 including a thermally conductive substrate including a liquid coolant passage for amplification of the nucleic acid, in accordance with the present disclosure. FIG. 1 illustrates an apparatus 100 from a top-down perspective on the top, and a cross-section perspective on the bottom viewed across line A-A.

In various examples, the apparatus 100 comprises an amplification chamber to contain a biochemical reaction associated with amplification of a biologic sample including a nucleic acid. As used herein, an amplification chamber refers to or includes an enclosed and/or semi-enclosed region of apparatus 100 that is capable of reaching appropriate thermal cycle temperatures for nucleic acid amplification. Referring to FIG. 1, the apparatus 100 comprises an amplification chamber 103. The amplification chamber 103 thickness may range between 5 micrometers (μm) and 100 μm. Also, amplification chamber 103 volume may vary between approximately 10 picoliters (pL) and 10 μL. As used herein, the terms “sample” and/or “biologic sample” generally refer to and/or include any material containing nucleic acid, including for example foods and allied products, clinical, and environmental samples. However, the sample will generally be a biological sample, which may contain any viral or cellular material, including all prokaryotic or eukaryotic cells, viruses, bacteriophages, mycoplasmas, protoplasts, and organelles. Additional examples of a biologic sample may include water, soil, surface swabs, fibers, and/or other environmental samples, as well as fruit juices, vegetable juices, and/or other food samples. Such biological material may thus comprise all types of mammalian and non-mammalian animal cells, plant cells, algae including blue-green algae, fungi, bacteria, protozoa, etc. Representative samples thus include whole blood and blood-derived products such as plasma, serum and buffy coat, urine, feces, cerebrospinal fluid or any other body fluids, tissues, cell cultures, cell suspensions, etc. The sample may comprise a lysate. The sample may also include relatively pure starting material such as a PCR product or semi-pure preparations obtained by other nucleic acid recovery processes.

In some examples, the apparatus further comprises a heater thermally coupled to the amplification chamber and to apply heat to the amplification chamber according to a heating and cooling protocol, the heating and cooling protocol associated with amplification of the nucleic acid. In some examples, the heater may be a resistor. In some examples, other heating elements may be used. Referring again to FIG. 1, the heater 105 may be thermally coupled to the amplification chamber 103 to apply heat to the amplification chamber according to a heating and cooling protocol, the heating and cooling protocol associated with amplification of the nucleic acid. As described herein, the heating and cooling protocol refers to or includes instructions for heating a biologic sample to an approximate temperature for an approximate amount of time and cooling the biologic sample to an approximate temperature for an approximate amount of time. The protocol may include a plurality of cycles of heating, a plurality of cycles of cooling, more cycles of heating than cooling, or more cycles of cooling than heating. A non-limiting example of a heating and cooling protocol includes a two-temperature protocol in which a sample is heated for 0.1 to 2 seconds at approximately 90° C. to 98° C. and then for 0.1 to 2 seconds at approximately 72° C., and repeated for 25-35 cycles. Additionally, heating and cooling protocols may vary based on the type of biologic sample, e.g., the type of nucleic acid being amplified, and each temperature may be approximate. Each respective heat cycle may be achieved by warming, e.g., heating, a heating element. A non-limiting example of a heating element used herein is a resistor. In some examples, the heater 105 is a thin-film resistor.

Referring to FIG. 1, apparatus 100 may include heater 105 to heat the biologic sample disposed in the amplification chamber 103. The heater 105 may heat the biologic sample in the amplification chamber 103 by application of a pulsed electric supply to the heater 105. The average power density applied to the heating element (e.g., heater 105) may be in the range of 10\hat{ }6-10\hat{ }8 W/m² (modelled 2.5×10\hat{ }6-1.3×10\hat{ }7 W/m²). This is an average power density, so the average power density may be reached by a pulse-width modulation technique. To operate, the heater 105 may be pulsed for a given time and then turned off. The pulse completely heats the biologic sample in the amplification chamber 103 to the denature temperature for amplification (e.g., approximately 95° C.). Responsive to active cooling, as discussed further herein, the apparatus 100 may cool down to the amplification chamber 103 anneal temperature by active cooling, then turn the heater 105 on again with different respective amplification (e.g., temperature). In some instances, the apparatus 100 may be communicatively coupled to a proportional-integral-derivative (PID) controller with a high-speed T-measurement sensor.

The apparatus may further include a thermally conductive substrate thermally coupled to the heater and including a liquid coolant passage to pass a liquid coolant through the thermally conductive substrate. As used herein, a thermally conductive substrate 101 refers to or includes a solid material including a channel, passage, via, tunnel, and/or other means for a liquid coolant pass through the solid material thereby rapidly cooling the solid material. Non-limiting examples of the thermally conductive substrate include silicon (Si), silicon nitride (SiN), silicon dioxide (SiO2), Silicon carbide (SiC), sapphire (Al2O3), copper, aluminum, steel, and other inorganic materials.

Referring again to FIG. 1, the apparatus 100 may include a thermally conductive substrate 101 thermally coupled to the heater 105 and including a liquid coolant passage 107 to pass a liquid coolant. The arrow on the left hand side of the illustration demonstrates the flow of liquid coolant. As the thermally conductive substrate 101 is described herein as being a solid material which permits passage of a liquid coolant, it is to be understood that the thermally conductive substrate 101 may have several parts that are arranged to permit the liquid coolant to pass there through. The following figures illustrate non-limiting example arrangements of the thermally conductive substrate 101. As illustrated in FIG. 1, the thermally conductive substrate 101 includes a first conductive layer 101-1 upon which the heater 105 is deposited, and a second conductive layer 101-2 which is a support substrate for the apparatus 100, and wherein the first conductive layer 101-1 and the second conductive layer 101-2 define the liquid coolant passage 107. Collectively, first conductive layer 101-1 and second conductive layer 101-2 are referred to herein as thermally conductive substrate 101. The thermally conductive substrate 101 separating the liquid coolant 107 and the PCR amplification chamber 103 may have a thickness between approximately 1 μm and 100 μm.

The liquid coolant may comprise water or any other suitable liquid coolant. In some examples, the liquid coolant may be 100% water. In some examples, the liquid coolant may include less than 100% water. In some examples, the liquid coolant may include 100% deionized water. In some examples, the liquid coolant may include an ethylene glycol and water solution. In some examples, the liquid coolant may include a propylene glycol and water solution. In some examples, the liquid coolant may include a dielectric fluid. Moreover, to increase the speed of amplification using apparatus 100, the liquid coolant may constantly or near constantly flow through the passage of the thermally conductive substrate 101. The liquid coolant flow rate may be in the range of approximately 0.1-10 millimeters per second (mm/s). While the liquid coolant may be constantly or near constantly flowing, during the heating phase of the heating and cooling protocol, enough power in the heater 105 may permit the biologic sample to heat up despite active cooling via the flow of the liquid coolant 107.

In some examples, the amplification chamber includes a lid comprising the thermally conductive substrate. For instance, referring again to FIG. 1, the amplification chamber 103 is defined, at least in part, by a lid 109. This lid may be comprised of any suitable material, and a non-limiting example material includes SU8.

In various examples, the apparatus 100 may be incorporated in a system for nucleic acid amplification. For instance, a biologic sample, such as a food sample, a clinical sample, or other sample described herein, may be input to a device input as described and illustrated herein. The device input may be provided on the apparatus 100 and/or as a separate component coupled to apparatus 100. The liquid coolant 107 may be provided by supply of liquid coolant that is fluidically coupled to apparatus 100, allowing for a continuous or near-continuous flow of liquid coolant 107 through apparatus 100 in the direction of the arrows shown in FIG. 1. In some examples, the liquid coolant 107 may form a closed loop such as when an outlet of the liquid coolant 107 is coupled to an inlet of the liquid coolant.

In some examples, the apparatus 100 may also be coupled to circuitry to control the heating and cooling of the amplification chamber 103. For instance, a controller and/or other form of circuitry may be coupled to the heater 105 to control the temperature of the heater 105. As a non-limiting example, the heater 105 may be a thin-film resistor, and a PID controller with a high-speed T-measurement sensor may be communicatively coupled to the thin-film resistor 105. The PID controller may provide a pulsed electric supply to the thin film resistor 105. Examples are not so limited. Additional and/or different types of controllers and/or circuitry may be coupled to additional and/or different types of heaters 105 and controlled to heat the amplification chamber 103.

Similarly, apparatus 100 may be coupled to additional components for sample testing and/or processing. As such, the amplification chamber 103 may include an ejection chamber disposed adjacent to the amplification chamber for ejecting the amplified nucleic acid from the amplification chamber 103. In some examples, the apparatus may be coupled to an additional component that ejects the amplified sample. In any scenario, the amplified sample may be ejected from apparatus 100 and/or by a system including apparatus 100 by an ejection chamber. The ejection chamber may include a drop-on-demand thermal bubble system including a thermal inkjet (TIJ) ejector. The TIJ ejector may implement a thermal resistor ejection element in an ejection chamber to a liquid sample and create bubbles that force the sample or other fluid drops out of the ejection chamber. In some examples, the amplified sample may be ejected from apparatus 100 and/or by a system including apparatus 100 by an ejection chamber that includes a drop-on-demand piezoelectric inkjet system including a piezoelectric inkjet (PIJ) ejector that implements a piezoelectric material actuator as an ejection element to generate pressure pulses that force liquid sample drops out of the ejection chamber. Examples are not so limited and additional and/or different types of ejectors may be used to eject sample from the amplification chamber. Similarly, different and/or additional components may be coupled to apparatus 100 to form a system for amplification of nucleic acids, as well as a system of purification, and a system for testing for nucleic acids of interest.

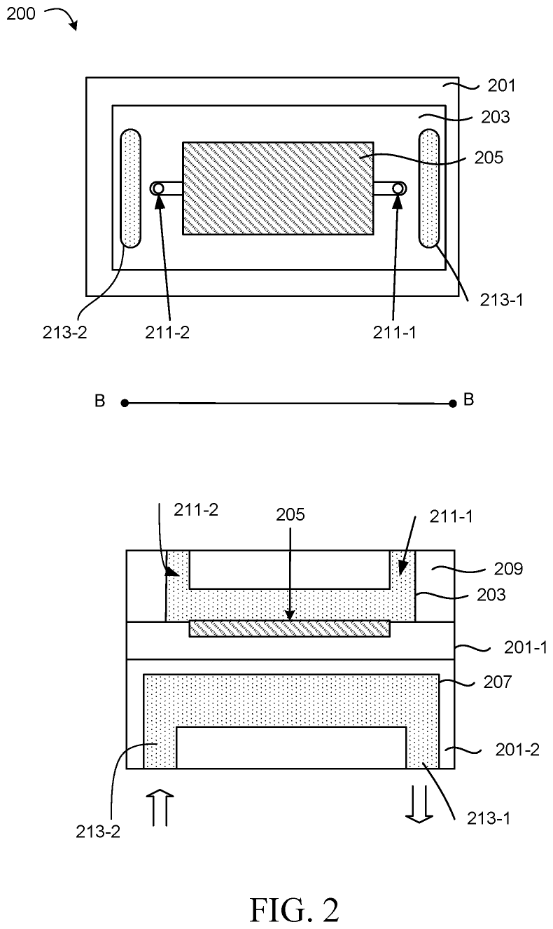

FIG. 2 illustrates an example apparatus 200 including a thermally conductive substrate including a liquid coolant passage for amplification of the nucleic acid and further illustrating a fluid inlet and a fluid outlet for the liquid coolant passage, in accordance with the present disclosure. FIG. 2 illustrates an apparatus 200 with a view of the apparatus 200 from the top down illustrated in the top of the page, and a view of the apparatus 200 from the line across B-B illustrated on the bottom half of the page. As illustrated in FIG. 1, arrows illustrate the flow of liquid coolant 207.

In various examples of the present disclosure, the apparatus may include a fluid inlet for the liquid coolant to enter and a fluid outlet for the liquid coolant to exit. The liquid coolant may enter the apparatus in various locations, and may exit the apparatus in various locations. Similarly, reagents and biologic samples may be input the apparatus in various locations, and in some instances removed. As such, the apparatus may include a reagent inlet for the reagent and/or biologic sample input and a reagent outlet for the reagent and/or biologic sample to exit. In some examples, the biologic sample may remain in the amplification chamber, though a vent may be used to vent air out of the amplification chamber, as discussed further herein. The reagent may include a plurality of components. Among these components are a nucleic acid template, such as a DNA template (e.g., double-stranded DNA) that contains a nucleic acid sequence to be amplified, an enzyme that polymerizes new nucleic acid strands (e.g., a polymerase enzyme such as DNA polymerase, e.g., Taq DNA polymerase), two nucleic acid primers (oligonucleotides, e.g., single-stranded) that are complementary to the 3′ (three prime) ends of each of the sense and antisense strands of the nucleic acid target, nucleoside triphosphates (NTPs) such as deoxyribonucleotide triphosphates (dNTPs) and ribonucleoside triphosphates (rNTPs), and a buffer. Specific buffer solutions may include bivalent cations, such as magnesium (Mg) or manganese (Mn) ions, and monovalent cations such as potassium (K) ions.

The apparatus illustrated in FIG. 2 illustrates many of the same components illustrated in FIG. 1 and is numbered accordingly. For instance, apparatus 200 includes a thermally conductive substrate 201, an amplification chamber 203, a lid 209, and a liquid coolant passage 207. Similarly, the apparatus 200 includes a heater 205. In some examples, the liquid coolant passage includes a liquid coolant inlet on a side of the apparatus opposite of the amplification chamber for the liquid coolant to enter the liquid coolant passage, and liquid coolant outlet on the side of the apparatus opposite of the amplification chamber for the liquid coolant to exit the liquid coolant passage. For instance, referring to FIG. 2, the apparatus 200 includes a liquid coolant inlet 213-2 on a side of the apparatus 200 distal to the amplification chamber 203 relative to the thermally conductive substrate 201. The liquid coolant inlet 213-2 may comprise a via or other passageway through which the liquid coolant may flow into the liquid coolant passage 207. The apparatus 200 may also include a liquid coolant outlet 213-1 on a side of the apparatus 200 distal to the amplification chamber 203 relative to the thermally conductive substrate 201. The liquid coolant outlet 213-1 may comprise a via or other passageway through which the liquid coolant may flow out of the liquid coolant passage 207. A section of thermally conductive substrate 201-2 may separate the liquid coolant inlet 213-2 and liquid coolant outlet 213-1.

As discussed with regards to FIG. 1, the heater 205 may be disposed on a thin layer of a thermally conductive substrate 201-1. A second part of the thermally conductive substrate, thermally conductive substrate 201-2, may form a supportive substrate 201-2 upon which the apparatus 200 is formed. Collectively, thermally conductive substrate 201-1 and thermally conductive substrate 201-2 are referred to herein as thermally conductive substrate 201.

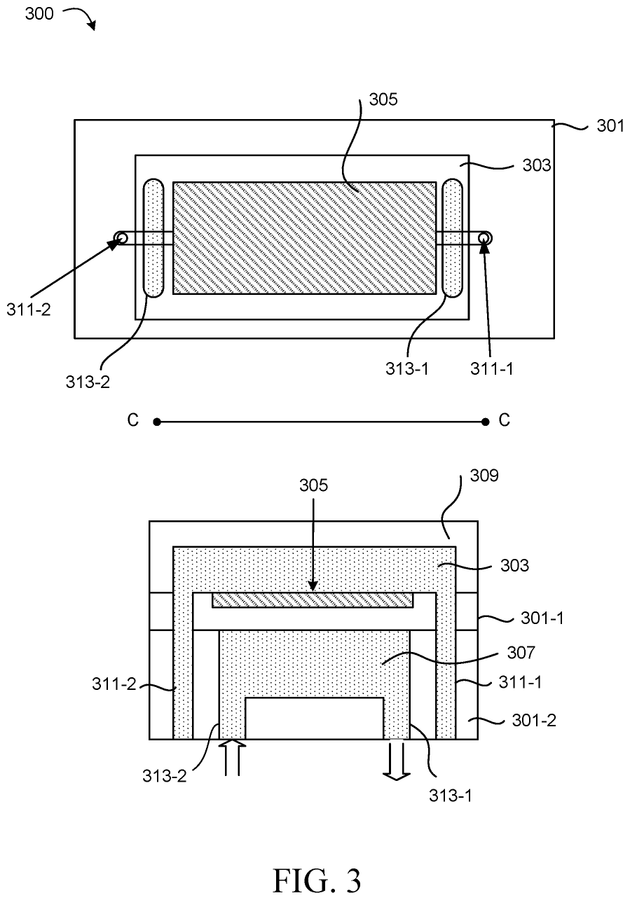

In some examples, the amplification chamber 203 includes a reagent chamber inlet or outlet 211-2 on a side of the apparatus 200 opposite of the liquid coolant passage 207 for the reagent and/or biologic sample to enter or exit the amplification chamber 203, and a reagent chamber inlet or outlet 211-1 on a side of the apparatus 200 opposite of the liquid coolant passage 207 for the reagent and/or biologic sample to enter or exit the amplification chamber 203. Different arrangements of the reagent input/output as well as liquid coolant input/output are contemplated. For instance, an example apparatus in which both reagent input/output and liquid coolant input/out are on a same respective side is discussed with regards to FIG. 3.

FIG. 3 illustrates an example apparatus 300 including a thermally conductive substrate including a liquid coolant passage for amplification of the nucleic acid in which a reagent input/output and a liquid coolant input/output are on a same side of the apparatus, in accordance with the present disclosure. FIG. 3 illustrates an apparatus 300 with a view of the apparatus 300 from the top down illustrated in the top of the page, and a view of the apparatus 300 from the line across C-C illustrated on the bottom half of the page.

The apparatus illustrated in FIG. 3 illustrates many of the same components illustrated in FIG. 1 and is numbered accordingly. For instance, apparatus 300 includes a thermally conductive substrate 301, an amplification chamber 303, a liquid coolant passage 307, and a lid 309. Similarly, the apparatus 300 includes a heater 305. In some examples, the liquid coolant passage includes a liquid coolant inlet 313-2 on a side of the apparatus opposite of the amplification chamber for the liquid coolant to enter the liquid coolant passage, and liquid coolant outlet 313-1 on the side of the apparatus opposite of the amplification chamber for the liquid coolant to exit the liquid coolant passage. A section of thermally conductive substrate 301-2 may separate the liquid coolant inlet 313-2 and liquid coolant outlet 313-1.

As discussed with regards to FIG. 1, the heater 305 may be disposed on a thin layer of a thermally conductive substrate 301-1. A second part of the thermally conductive substrate, thermally conductive substrate 301-2, may form a supportive substrate 301-2 upon which the apparatus 300 is formed. Collectively, thermally conductive substrate 301-1 and thermally conductive substrate 301-2 are referred to herein as thermally conductive substrate 301.

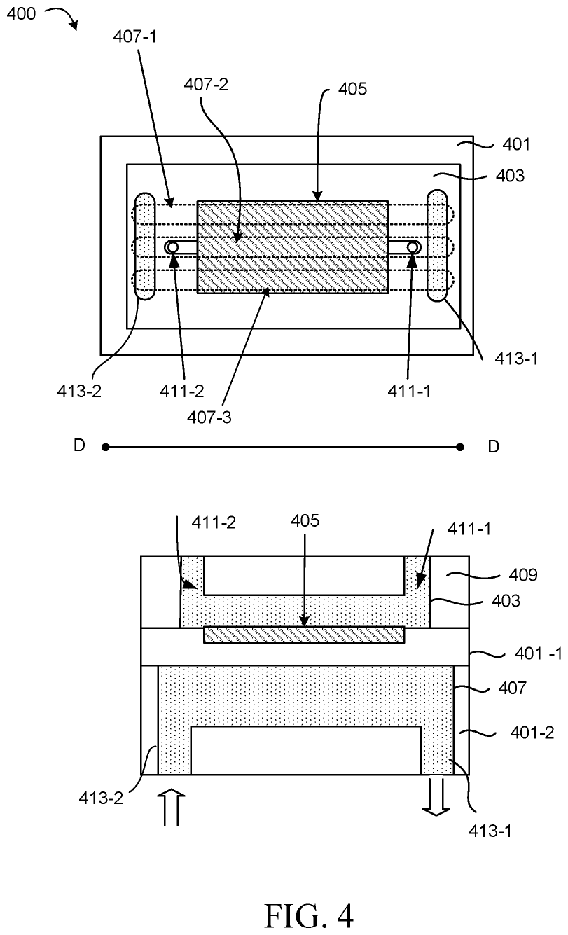

In some examples, the amplification chamber 303 includes a reagent chamber inlet or outlet 311-2 on a same side of the apparatus 300 opposite of the liquid coolant passage 307 for the reagent and/or biologic sample to enter or exit the amplification chamber 303, and a reagent chamber inlet or outlet 311-1 on a side of the apparatus 300 opposite of the liquid coolant passage 307 for the reagent and/or biologic sample to enter or exit the amplification chamber 303. Different arrangements of the reagent input/output as well as liquid coolant input/output are contemplated. For instance, an example apparatus with multiple liquid coolant channels is discussed with regards to FIG. 4.

FIG. 4 illustrates an example apparatus 400 including a thermally conductive substrate and a plurality of liquid coolant passages for amplification of a nucleic acid, in accordance with the present disclosure. FIG. 4 illustrates an apparatus 400 with a view of the apparatus 400 from the top down illustrated in the top of the page, and a view of the apparatus 400 from the line across D-D illustrated on the bottom half of the page.

The apparatus illustrated in FIG. 4 illustrates many of the same components illustrated in FIG. 1 and is numbered accordingly. For instance, apparatus 400 includes a thermally conductive substrate 401, an amplification chamber 403, a liquid coolant passage 407, and a lid 409. Similarly, the apparatus 400 includes a heater 405, a reagent chamber inlet or outlet 411-2 on a side of the apparatus 400 opposite of the liquid coolant passage 407 for the reagent and/or biologic sample to enter or exit the amplification chamber 403, and a reagent chamber inlet or outlet 411-1 on a side of the apparatus 400 opposite of the liquid coolant passage 407 for the reagent and/or biologic sample to enter or exit the amplification chamber 403. In some examples, the liquid coolant passage includes a liquid coolant inlet 413-2 on a side of the apparatus opposite of the amplification chamber for the liquid coolant to enter the liquid coolant passage, and liquid coolant outlet 413-1 on the side of the apparatus opposite of the amplification chamber for the liquid coolant to exit the liquid coolant passage. A section of thermally conductive substrate 401-2 may separate the liquid coolant inlet 413-2 and liquid coolant outlet 413-1.

As discussed with regards to FIG. 1, the heater 405 may be disposed on a thin layer of a thermally conductive substrate 401-1. A second part of the thermally conductive substrate, thermally conductive substrate 401-2, may form a supportive substrate 401-2 upon which the apparatus 400 is formed. Collectively, thermally conductive substrate 401-1 and thermally conductive substrate 401-2 are referred to herein as thermally conductive substrate 401.

As illustrated in FIG. 4, a plurality of cooling chambers 407-1, 407-2, and 407-3 may be used to more rapidly cool the biologic sample between heating cycles. As illustrated in the top-down illustration, the amplification chamber 403 extends across all of the plurality of cooling chambers 407-1, 407-2, and 407-3. Each of the cooling chambers 407-1, 407-2, and 407-3 may have a different respective liquid coolant inlet and a different respective liquid coolant outlet. Examples are not so limited, however, and each of the cooling chambers 407-1, 407-2, and 407-3 may have a different respective liquid coolant inlet and a same liquid coolant outlet, and/or each of the cooling chambers 407-1, 407-2, and 407-3 may have a same liquid coolant inlet and a different respective liquid coolant outlet, and/or each of the cooling chambers 407-1, 407-2, and 407-3 may have a same liquid coolant inlet and a same liquid coolant outlet.

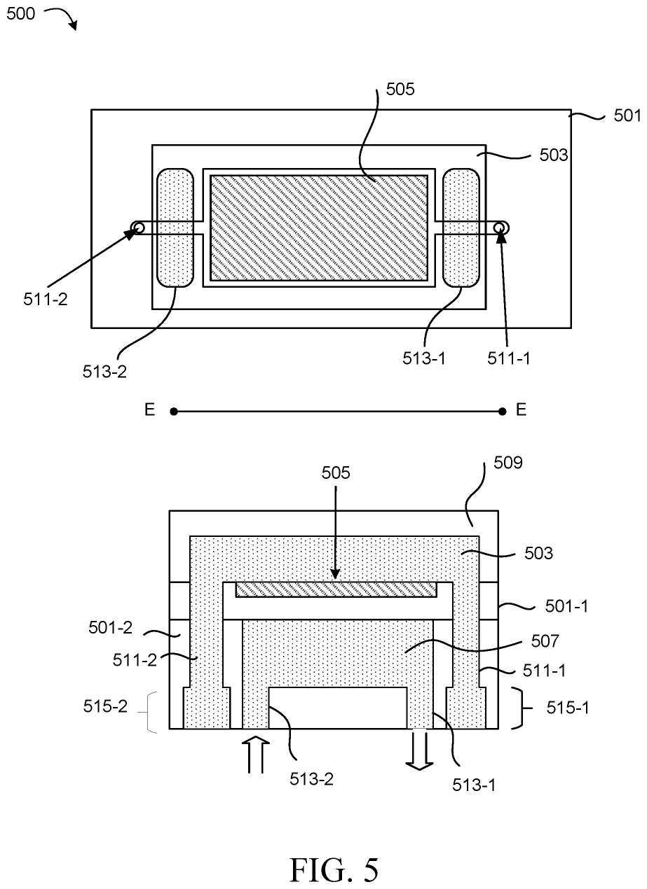

Different arrangements of the reagent input/output as well as liquid coolant input/output are contemplated. For instance, an example apparatus with interposer liquid coolant channels is discussed with regards to FIG. 5.

FIG. 5 illustrates an example apparatus 500 including a thermally conductive substrate and a plurality of liquid coolant passages for amplification of a nucleic acid, in accordance with the present disclosure. FIG. 5 illustrates an apparatus 500 with a view of the apparatus 500 from the top down illustrated in the top of the page, and a view of the apparatus 500 from the line across E-E illustrated on the bottom half of the page.

The apparatus illustrated in FIG. 5 illustrates many of the same components illustrated in FIG. 1 and is numbered accordingly. For instance, apparatus 500 includes a thermally conductive substrate 501, an amplification chamber 503, a liquid coolant passage 507, and a lid 509. Similarly, the apparatus 500 includes a heater 505. In some examples, the liquid coolant passage includes a liquid coolant inlet 513-2 on a side of the apparatus opposite of the amplification chamber for the liquid coolant to enter the liquid coolant passage, and liquid coolant outlet 513-1 on the side of the apparatus opposite of the amplification chamber for the liquid coolant to exit the liquid coolant passage. A section of thermally conductive substrate 501-2 may separate the liquid coolant inlet 513-2 and liquid coolant outlet 513-1.

As discussed with regards to FIG. 1, the heater 505 may be disposed on a thin layer of a thermally conductive substrate 501-1. A second part of the thermally conductive substrate, thermally conductive substrate 501-2, may form a supportive substrate 501-2 upon which the apparatus 500 is formed. Collectively, thermally conductive substrate 501-1 and thermally conductive substrate 501-2 are referred to herein as thermally conductive substrate 501.

In some examples, the amplification chamber 503 includes a reagent chamber inlet or outlet 511-2 on a same side of the apparatus 500 opposite of the liquid coolant passage 507 for the reagent and/or biologic sample to enter or exit the amplification chamber 503, and a reagent chamber inlet or outlet 511-1 on a side of the apparatus 500 opposite of the liquid coolant passage 507 for the reagent and/or biologic sample to enter or exit the amplification chamber 503. As illustrated in FIG. 5, the reagent inlet or outlet 511-1 and reagent inlet or outlet 511-2 may each include an interposer. For instance, reagent inlet/outlet 511-2 includes interposer 515-2 and reagent inlet/outlet 511-1 includes interposer 515-1.

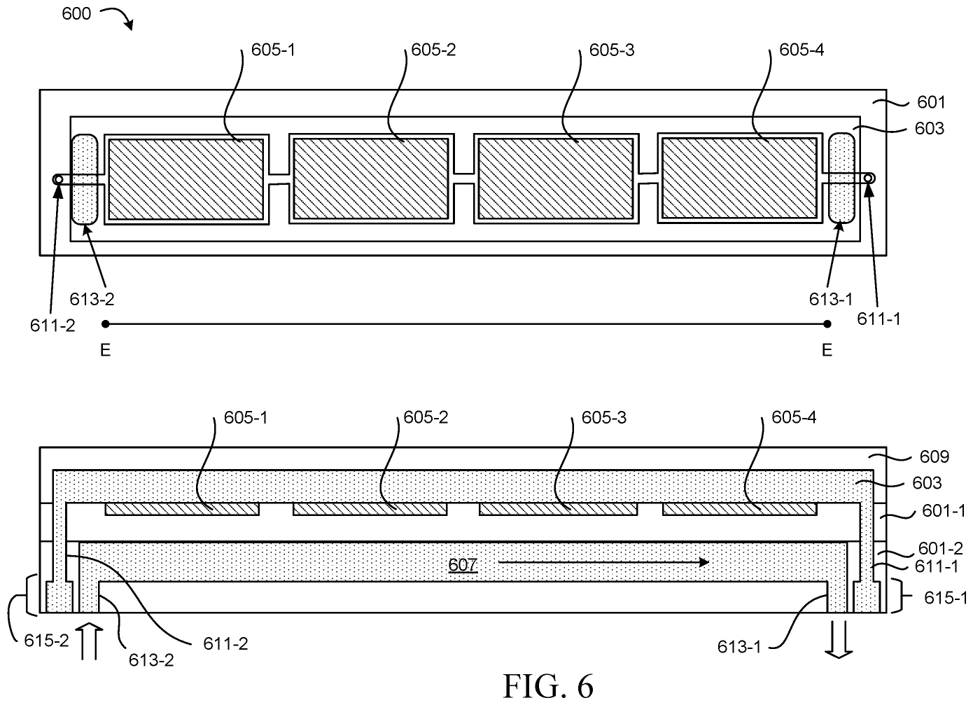

Different arrangements of the reagent input/output as well as liquid coolant input/output are contemplated. For instance, an example apparatus with multiple amplification chambers is discussed with regards to FIG. 6.

FIG. 6 illustrates an example apparatus 600 including a thermally conductive substrate and a plurality of amplification chambers for amplification of a nucleic acid, in accordance with the present disclosure. FIG. 6 illustrates an apparatus 600 with a view of the apparatus 600 from the top down illustrated in the top of the page, and a view of the apparatus 600 from the line across E-E illustrated on the bottom half of the page.

Various examples of the present disclosure include an apparatus, comprising an amplification chamber to contain a biochemical reaction associated with amplification of a biologic sample including a nucleic acid. The apparatus may also include a series of heaters thermally coupled to the amplification chamber and to apply heat to the amplification chamber according to a heating and cooling protocol, the heating and cooling protocol associated with amplification of the nucleic acid. For instance, the apparatus 600 may include a series of heater s 605-1, 605-2, 605-3, and 605-4, and a lid 609. Although four heaters are illustrated, more and/or fewer heaters may be included. Also, the heaters may or may not be of the same size or shape. The series of heaters may be thermally coupled to the amplification chamber 603 according to a heating and cooling protocol, the heating and cooling protocol associated with amplification of the nucleic acid.

Similar to examples discussed herein, the apparatus may include a thermally conductive substrate in contact with the series of heaters and including a liquid coolant passage to pass a liquid coolant through the thermally conductive substrate. For instance, referring again to FIG. 6, the apparatus 600 may include a thermally conductive substrate 601 in contact with the heaters 605-1, 605-2, 605-3, 605-4 (collectively referred to herein as heaters 605) and including a liquid coolant passage 607 to pass a liquid coolant. As the thermally conductive substrate 601 is described herein as being a solid material which permits passage of a liquid coolant, it is to be understood that the thermally conductive substrate 601 may have several parts that are arranged to permit the liquid coolant to pass there through. The following figures illustrate non-limiting example arrangements of the thermally conductive substrate 601. As illustrated in FIG. 6, the thermally conductive substrate 601 includes a first conductive layer 601-1 upon which the heaters 605 are deposited, and a second conductive layer 601-2 which is a support substrate for the apparatus 600, and wherein the first conductive layer 601-1 and the second conductive layer 601-2 define the liquid coolant passage 607. The thermally conductive substrate 601 separating the liquid coolant 607 and the PCR amplification chamber 603 may have a thickness between approximately 1 μm and 100 μm.

Various examples include different arrangements for liquid coolant input and/or exit. For instance, in the example illustrated in FIG. 6 the amplification chamber 603 includes a reagent chamber inlet or outlet 611-2 on a same side of the apparatus 600 as the liquid coolant passage 607 relative to the amplification chamber, and a reagent chamber inlet or outlet 611-1 on a same side of the apparatus 600 as the liquid coolant passage 607 relative to the amplification chamber. The reagent and/or biologic sample may enter and/or exit the amplification chamber 603 via the reagent chamber inlet or outlet 611-1 and 611-2. As illustrated in FIG. 6, the reagent inlet or outlet 611-1 and reagent inlet or outlet 611-2 may each include an interposer. For instance, reagent inlet/outlet 611-2 includes interposer 615-2 and reagent inlet/outlet 611-1 includes interposer 615-1.

In some examples, the liquid coolant passage 607 includes a liquid coolant inlet 613-2 on a side of the apparatus 600 opposite of the amplification chamber 603 for the liquid coolant to enter the liquid coolant passage 607, and liquid coolant outlet 613-1 on the side of the apparatus 600 opposite of the amplification chamber 603 for the liquid coolant to exit the liquid coolant passage 607. A section of thermally conductive substrate 601-2 may separate the liquid coolant inlet 613-2 and liquid coolant outlet 613-1. In various examples, the liquid coolant inlet 613-2 and liquid coolant outlet 613-1 may be on a same side of the apparatus 600 as the reagent inlet/outlet 611-2 and the reagent inlet/outlet 611-1, as illustrated in FIG. 6. However, examples are not so illustrated, and other arrangements are contemplated.

In some examples, the thermally conductive substrate further defines a liquid coolant inlet on a side of the apparatus that is opposite of the series of heaters and a liquid coolant outlet on the side of the apparatus that is opposite of the series of heaters, such that the liquid coolant flows in a direction parallel to the series of heaters from the liquid coolant inlet to the liquid coolant outlet. For instance, the thermally conductive substrate 601, comprising substrate 601-1 and substrate 601-2 defines a liquid coolant inlet 613-2 on a side of the apparatus 600 that is opposite of the series of heaters 605 and a liquid coolant outlet 613-1 on a side of the apparatus that is opposite of the series of the heaters 605, such that the liquid coolant flows in a direction parallel to the series of heaters as indicated by the arrow in liquid coolant passage 607.

In various examples, the series of heaters may be pulsed as a group, such that each of the plurality of heaters reach a same temperature together. In some examples, each of the series of heaters is independently pulsed for an amount of time for the biochemical reaction. For instance, heater 605-1 may be set to pulse at a first temperature for PCR amplification, whereas heater 605-2 may be set to pulse at a second temperature for PCR amplification, whereas heater 605-3 may be set to pulse at a third temperature for PCR amplification, and so forth.

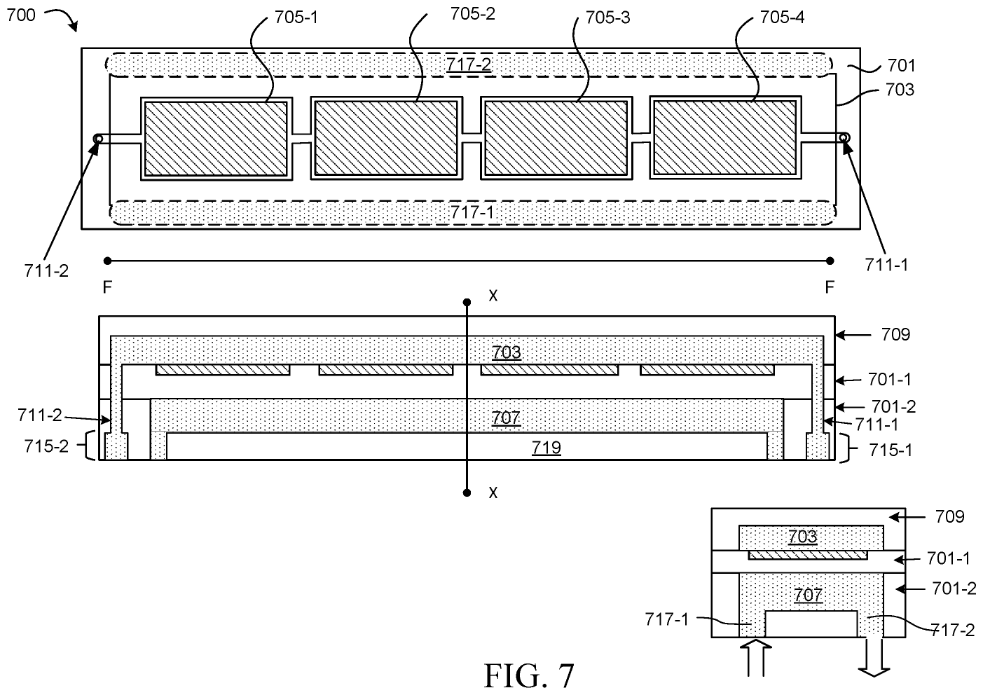

FIG. 7 illustrates an example apparatus 700 including a thermally conductive substrate, a plurality of amplification chambers for amplification of a nucleic acid, and liquid coolant inlet troughs in accordance with the present disclosure. FIG. 7 illustrates an apparatus 700 with a view of the apparatus 700 from the top down illustrated in the top of the page, a view of the apparatus 700 from the line across F-F illustrated on the bottom half of the page, and a cross-section across line X-X in the bottom right part of the page.

The apparatus illustrated in FIG. 7 illustrates many of the same components illustrated in FIG. 6 and is numbered accordingly. For instance, apparatus 700 includes a thermally conductive substrate 701, an amplification chamber 703, a liquid coolant passage 707, and a lid 709. Similarly, the apparatus 700 includes a series of heaters 705-1, 705-2, 705-3, and 705-4.

In some examples, the thermally conductive substrate further defines a liquid coolant inlet trough disposed parallel to the series of heaters and a liquid coolant outlet trough disposed parallel to the series of heaters, such that the liquid coolant flows in a direction orthogonal to the series of heaters. For instance, referring to FIG. 7, the thermally conductive substrate 701-1 and 701-2 may define a liquid coolant inlet trough 717-1 disposed parallel to the series of heaters 705-1, 705-2, 705-3, 705-4, and a liquid coolant outlet trough 717-2 disposed parallel to the series of heaters 705-1, 705-2, 705-3, 705-4, such that the liquid coolant flows in a direction orthogonal to the series of heaters 705-1, 705-2, 705-3, 705-4, as indicated by the arrow pointing toward the top of the page. A cross-sectional diagram of the flow of the apparatus 700 is also illustrated in the bottom corner of the page, illustrating the flow of the liquid coolant through the bottom of the apparatus, orthogonal to the apparatus, and out through the bottom of the apparatus to the exit of the apparatus again. In some examples, the apparatus 700 may include a reagent inlet or outlet 711-1 and reagent inlet or outlet 711-2 may each include an interposer. For instance, reagent inlet/outlet 711-2 includes interposer 715-2 and reagent inlet/outlet 711-1 includes interposer 715-1. An additional interposer may be utilized. For instance, interposer 719 may be included at the base of the apparatus 700.

Although examples herein describe the flow of liquid coolant in a relatively direct manner, examples are not so limited. For instance, in some examples, the flow of the liquid coolant may be in a serpentine, chevron, or other non-linear manner. As such, the thermally conductive substrate may be arranged in any non-linear arrangement to direct the liquid coolant to flow in such directions and to increase the rate of cooling for nucleic acid amplification, as discussed herein. As such, in some examples, the thermally conductive substrate includes channel separators to direct the liquid coolant flow in a serpentine direction from a liquid coolant inlet to a liquid coolant outlet.

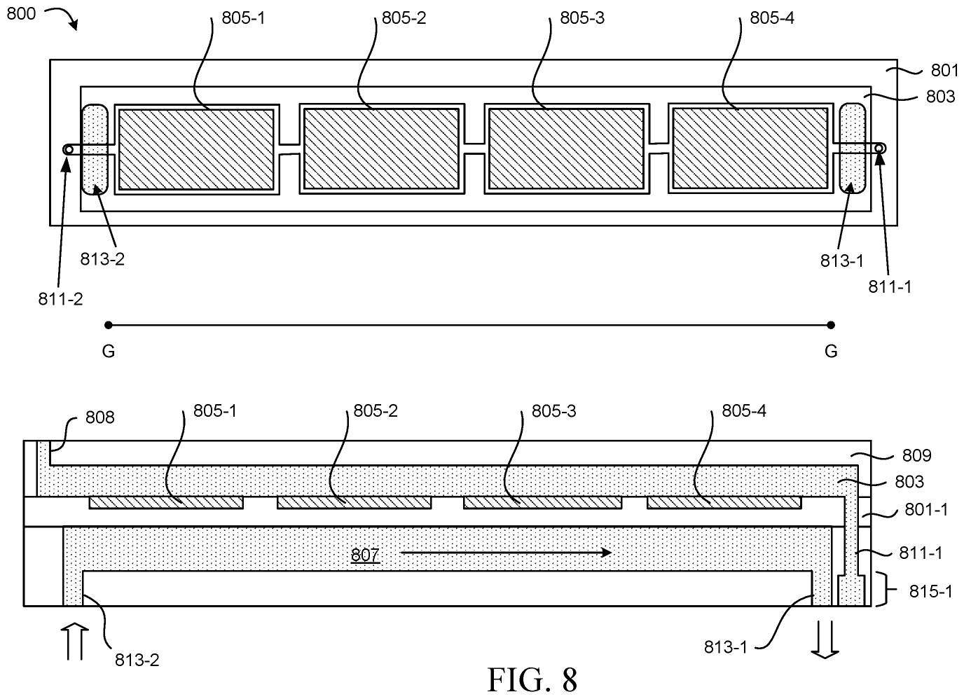

FIG. 8 illustrates an example apparatus 800 including a plurality of amplification chambers for amplification of a nucleic acid and a venting port, in accordance with the present disclosure. FIG. 8 illustrates an apparatus 800 with a view of the apparatus 800 from the top down illustrated in the top of the page, and a view of the apparatus 800 from the line across G-G illustrated on the bottom half of the page.

The apparatus illustrated in FIG. 8 illustrates many of the same components illustrated in FIG. 7 and is numbered accordingly. For instance, apparatus 800 includes a thermally conductive substrate 801, an amplification chamber 803, a liquid coolant passage 807, and a lid 809. Similarly, the apparatus 800 includes a series of heaters 805-1, 805-2, 805-3, and 805-4 (referred to collectively as the series of heaters 805). As illustrated in FIG. 8, the thermally conductive substrate 801 includes a first conductive layer 801-1 upon which the series of heaters 805 are deposited, and a second conductive layer 801-2 which is a support substrate for the apparatus 800, and wherein the first conductive layer 801-1 and the second conductive layer 801-2 define the liquid coolant passage 807.

The apparatus 800 includes a liquid coolant inlet 813-2 on a side of the apparatus 800 opposite of the amplification chamber 803 for the liquid coolant to enter the liquid coolant passage 807, and liquid coolant outlet 813-1 on the side of the apparatus 800 opposite of the amplification chamber 803 for the liquid coolant to exit the liquid coolant passage 807.

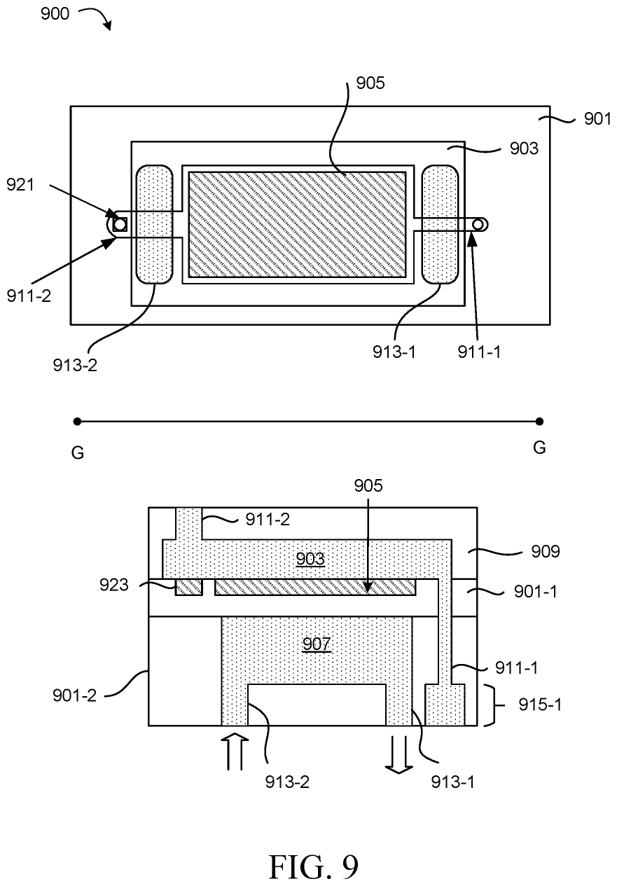

In some examples, the apparatus 800 may include a reagent inlet or outlet 811-1 and interposer 815-1. Priming of the apparatus 800 with reagent and/or reagents may be done via capillary force and venting port or plurality of venting ports. In various examples, the apparatus 800 may include a venting port 808 or plurality of venting ports. In some examples, venting port 808 (or plurality of venting ports as applicable) may be collocated with a heater such as a TIJ resistor (or any other drop ejecting device) to eject amplified nucleic acid from the apparatus 800 for further analysis, such as is illustrated in FIG. 9.

FIG. 9 illustrates an example apparatus 900 including a jetting nozzle-venting port, in accordance with the present disclosure. FIG. 9 illustrates an apparatus 900 with a view of the apparatus 900 from the top down illustrated in the top of the page, and a view of the apparatus 900 from the line across G-G illustrated on the bottom half of the page.

The apparatus illustrated in FIG. 9 illustrates many of the same components illustrated in FIG. 5 and is numbered accordingly. For instance, apparatus 900 includes a thermally conductive substrate 901, an amplification chamber 903, a liquid coolant passage 907, and a lid 909. Similarly, the apparatus 900 includes a heater 905. In some examples, the liquid coolant passage includes a liquid coolant inlet 913-2 on a side of the apparatus opposite of the amplification chamber 903 for the liquid coolant to enter the liquid coolant passage, and liquid coolant outlet 913-1 on the side of the apparatus opposite of the amplification chamber 903 for the liquid coolant to exit the liquid coolant passage. A section of thermally conductive substrate 901-2 may separate the liquid coolant inlet 913-2 and liquid coolant outlet 913-1.

As discussed with regards to FIG. 1, the heater 905 may be disposed on a thin layer of a thermally conductive substrate 901-1. A second part of the thermally conductive substrate, thermally conductive substrate 901-2, may form a supportive substrate 901-2 upon which the apparatus 900 is formed. Collectively, thermally conductive substrate 901-1 and thermally conductive substrate 901-2 are referred to herein as thermally conductive substrate 901.

In some examples, the amplification chamber 903 includes a reagent chamber inlet or outlet 911-1 on a same side of the apparatus 900 as the liquid coolant passage 907 for the reagent and/or biologic sample to enter or exit the amplification chamber 903, and a reagent chamber inlet or outlet 911-2 on a side of the apparatus 900 opposite of the liquid coolant passage 907 for the reagent and/or biologic sample to enter or exit the amplification chamber 903. As illustrated in FIG. 9, the reagent inlet or outlet 911-1 may include an interposer 915-1.

The reagent chamber inlet or outlet 911-2 may also have a nozzle with a resistor that may be used to eject amplified product from the amplification chamber 903. For instance, TIJ resistor 923, working as a venting port during initial priming, may be used with nozzle 921 to eject amplified nucleic acids from the amplification chamber 903 further analysis. As such, apparatus 900 may be used to analyze nucleic acid samples while apparatus 900 is still working to amplify nucleic acids received via the reagent chamber inlet or outlet 911-1.

Although figures and examples herein describe apparatus in which a chamber shape is generally square and amplification chamber size is generally larger than the heater size, examples are not so limited. For instance, the amplification chamber size may be smaller than the heater area size, thereby improving temperature uniformity across the amplification chamber. Additionally, the shape of the amplification chamber may be different than the shape of the heater. For example, the amplification chamber may be rectangular, square, oval, circular, rhomboidal, and/or any other shape.

In various examples, the apparatus may include multiple amplification chambers, and the plurality of amplification chambers may be interconnected with each other for priming and reagent delivery. The connecting bridges between amplification chambers may be formed by silicon, SU8, or other suitable material, and may have different size and/or shape properties to avoid capillary breaks.

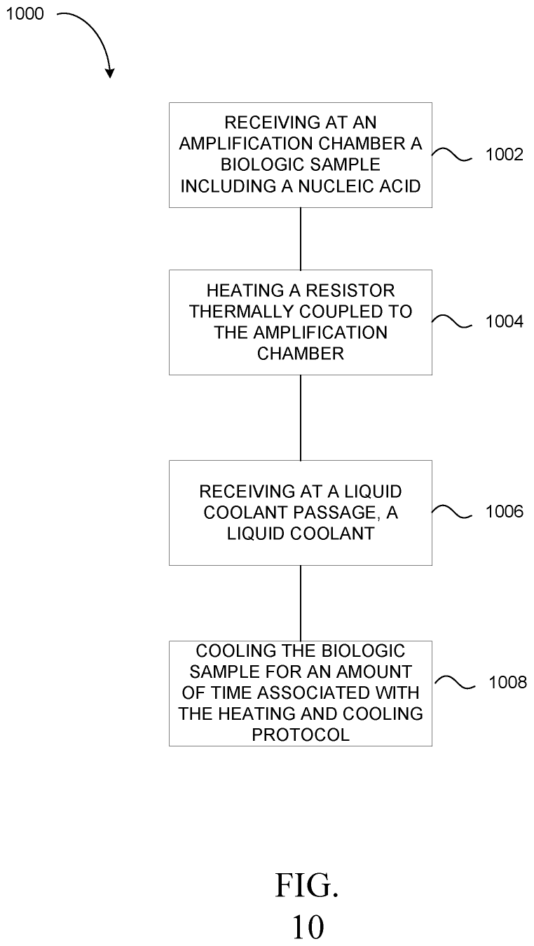

FIG. 10 illustrates an example method 1000 for amplification of a nucleic acid, in accordance with the present disclosure. As described herein, the method 1000 includes receiving at an amplification chamber defined by a thermally conductive substrate, a biologic sample including a nucleic acid at 1002. The method also includes heating a heater thermally coupled to the amplification chamber to a temperature of a heating and cooling protocol, the heating and cooling protocol associated with amplification of the nucleic acid at 1004.

At 1006 the method includes receiving at a liquid coolant passage defined by the thermally conductive substrate and in contact with the amplification chamber, a liquid coolant. At 1008 the method includes cooling the biologic sample for an amount of time associated with the heating and cooling protocol.

In some examples, the method 1000 includes receiving the biologic sample at a sample inlet and discharging the biologic sample at a sample outlet, the biologic sample flowing along a first plane, and receiving the liquid coolant includes receiving the liquid coolant at a liquid coolant inlet of the liquid coolant passage and discharging the liquid coolant at a liquid coolant outlet of the liquid coolant passage, the liquid coolant flowing along a second plane orthogonal to the first plane.

In some examples, receiving the biologic sample includes receiving the biologic sample at a sample inlet and discharging the biologic sample at a sample outlet, the biologic sample flowing along a first plane, and receiving the liquid coolant includes receiving the liquid coolant at a liquid coolant inlet of the liquid coolant passage and discharging the liquid coolant at a liquid coolant outlet of the liquid coolant passage, the liquid coolant flowing along a second plane parallel to the first plane.

In some examples, heating the heater includes heating a plurality of heaters arranged serially and thermally coupled to the amplification chamber, wherein each respective heater is warmed to a different respective temperature of the heating and cooling protocol. In some examples, the method includes ejecting the biologic sample from the amplification via an ejection chamber disposed adjacent to the amplification chamber. For instance, the nozzle 911-2 illustrated in FIG. 9 may form an ejection chamber, and using heater 923, the biologic sample may be ejected from the amplification chamber.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

### Experimental/More Detailed Examples

As further illustrated below in connection with the examples described herein, modeling was used to simulate performance of the example apparatus for nucleic acid amplification. Specifically, ANSYS modeling was used to analyze the thermal models described herein.

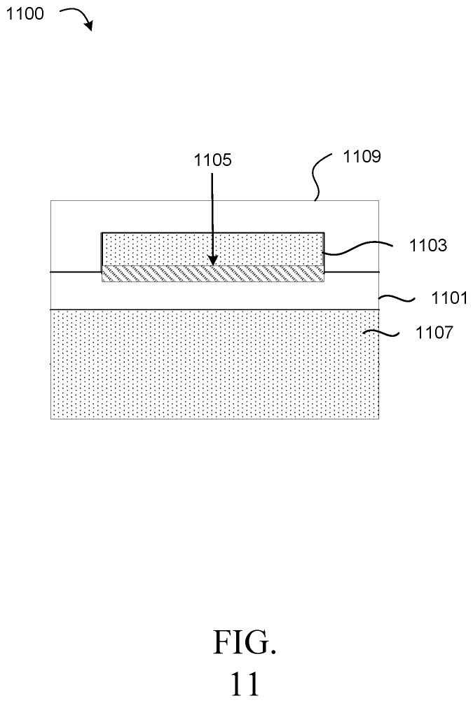

FIG. 11 illustrates a block diagram of an example apparatus used during a feasibility experiment. For the ANSYS modeling, an apparatus similar to apparatus 1100 illustrated in FIG. 11 was constructed. Apparatus 11 included an amplification fluid layer 1103, a resistor 1105, silicon 1101, flowing fluid 1107, and an SU8 lid 1109. The resistor and amplification fluid were 100 μm wide and the overall length of the silicon in the flow direction was 300 μm. The depth into the page for this new geometric configuration was 1 mm.

The apparatus 11 was run over a range of flowing fluid velocities, 0.1 mm/s, 1 mm/s and 10 mm/s. The resistor provided heat at a constant power level for 50 ms followed by a cooldown portion for an additional 100 ms with the flowing fluid moving during the entire simulation. The power level was set based on keeping the amplification fluid just above the resistor around approximately 100° C. The power level was set to 0.25 W. The resistor was allowed to heat for 50 ms but the cooling was run for 150 ms.

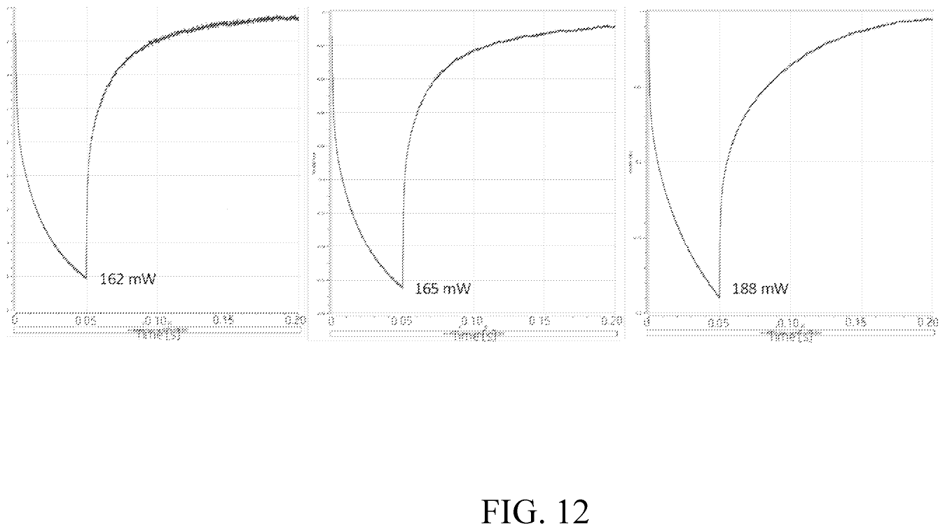

FIG. 12 illustrates peak heat fluxes to the amplification fluid at 162, 165, and 188 mW for liquid coolant flows, in accordance with the present disclosure. Specifically, FIG. 12 illustrates the volume averaged amplification fluid temperatures (y axis) as a function of time (x axis) at a liquid coolant flow rate of 0.1 millimeter per second (mm/s) on the left, at a liquid coolant flow rate of 1 mm/s in the middle, and at a liquid coolant flow rate of 10 mm/s on the right. The heat flux was measured. As used herein, heat flux refers to or includes the rate of heat energy transferred through a given surface per unit surface. As such, the examples illustrated that when the liquid coolant had a flow rate of 0.1 mm/s through the apparatus 1100 illustrated in FIG. 11, a heat flux of 162 mW was measured in amplification chamber 1103 at 0.05 seconds; when the liquid coolant had a flow rate of 1 mm/s through the apparatus 1100 illustrated in FIG. 11, a heat flux of 165 mW was measured in amplification chamber 1103 at 0.05 seconds; and when the liquid coolant had a flow rate of 10 mm/s through the apparatus 1100 illustrated in FIG. 11, a heat flux of 188 mW was measured in amplification chamber 1103 at 0.05 seconds. These data illustrate a strong influence on cooling of the amplification chamber and flow of the liquid coolant, even to the point of limiting the peak temperature during heating at the highest velocity of the liquid coolant. At the 1 mm/s liquid coolant velocity, the entire amplification fluid volume met the criteria to be active in less than 200 ms. The results demonstrated in FIG. 12 illustrated that rapid PCR processing based on thin film resistors, as discussed herein, may drastically reduce cooling times. The results illustrated improved PCR processing times even without optimizing the timing of the heating and cooling events. The results demonstrate that despite the large differences in temperature between the 0 mm/s, 1 mm/s, and 10 mm/s cases, there is a very small difference in heat flux. Peak heat fluxes to the fluid are 0.162, 0.165, and 0.188 W respectively. The 3 mW difference between no flow and 1e-3 mm/s causes an 8° C. difference at 200 ms.

1. An apparatus, comprising:
an amplification chamber to contain a biochemical reaction associated with amplification of a biologic sample including a nucleic acid;
a heater thermally coupled to the amplification chamber; and
a thermally conductive substrate in contact with the heater and including a liquid coolant passage to pass a liquid coolant through the thermally conductive substrate.

2. The apparatus of claim 1, wherein the thermally conductive substrate includes a first conductive layer upon which the heater is deposited, and a second conductive layer which is a support substrate for the apparatus, and wherein the first conductive layer and the second conductive layer define the liquid coolant passage.

3. The apparatus of claim 1, wherein the amplification chamber includes a lid comprising the thermally conductive substrate, and the thermally conductive substrate includes silicon.

4. The apparatus of claim 1, wherein the liquid coolant passage includes a liquid coolant inlet on a side of the apparatus opposite of the amplification chamber for the liquid coolant to enter the liquid coolant passage, and liquid coolant outlet on the side of the apparatus opposite of the amplification chamber for the liquid coolant to exit the liquid coolant passage.

5. The apparatus of claim 1, wherein the heater is a thin-film resistor.

6. An apparatus, comprising:
an amplification chamber to contain a biochemical reaction associated with amplification of a biologic sample including a nucleic acid;
a series of heaters thermally coupled to the amplification chamber; and
a thermally conductive substrate in contact with the series of heaters and including a liquid coolant passage to pass a liquid coolant through the thermally conductive substrate.

7. The apparatus of claim 6, wherein the thermally conductive substrate further defines a liquid coolant inlet on a side of the apparatus that is opposite of the series of heaters and a liquid coolant outlet on the side of the apparatus that is opposite of the series of heaters, such that the liquid coolant flows in a direction parallel to the series of heaters from the liquid coolant inlet to the liquid coolant outlet.

8. The apparatus of claim 6, wherein the thermally conductive substrate further defines a liquid coolant inlet trough disposed parallel to the series of heaters and a liquid coolant outlet trough disposed parallel to the series of heaters, such that the liquid coolant flows in a direction orthogonal to the series of heaters.

9. The apparatus of claim 6, wherein each of the series of heaters is independently pulsed for an amount of time for the biochemical reaction.

10. The apparatus of claim 6, wherein the thermally conductive substrate includes channel separators to direct the liquid coolant flow in a serpentine direction from a liquid coolant inlet to a liquid coolant outlet.

11. A method, comprising:
receiving at an amplification chamber defined by a thermally conductive substrate, a biologic sample including a nucleic acid;
heating a heater thermally coupled to the amplification chamber to a temperature of a heating and cooling protocol, the heating and cooling protocol associated with amplification of the nucleic acid;
receiving at a liquid coolant passage defined by the thermally conductive substrate and in contact with the amplification chamber, a liquid coolant; and
cooling the biologic sample for an amount of time associated with the heating and cooling protocol.

12. The method of claim 11, wherein:
receiving the biologic sample includes receiving the biologic sample at a sample inlet and discharging the biologic sample at a sample outlet, the biologic sample flowing along a first plane, and
receiving the liquid coolant includes receiving the liquid coolant at a liquid coolant inlet of the liquid coolant passage and discharging the liquid coolant at a liquid coolant outlet of the liquid coolant passage, the liquid coolant flowing along a second plane orthogonal to the first plane.

13. The method of claim 11, wherein:
receiving the biologic sample includes receiving the biologic sample at a sample inlet and discharging the biologic sample at a sample outlet, the biologic sample flowing along a first plane, and
receiving the liquid coolant includes receiving the liquid coolant at a liquid coolant inlet of the liquid coolant passage and discharging the liquid coolant at a liquid coolant outlet of the liquid coolant passage, the liquid coolant flowing along a second plane parallel to the first plane.

14. The method of claim 11, wherein heating the heater includes heating a plurality of heaters arranged serially and thermally coupled to the amplification chamber, wherein each respective heater is warmed to a different respective temperature of the heating and cooling protocol.

15. The method of claim 11, further including ejecting the biologic sample from the amplification via an ejection chamber disposed adjacent to the amplification chamber.
